(19) United States
(12) Patent Application Publication
(10) Pub. No.: US 2016/0235358 A1
(43) Pub. Date: Aug. 18, 2016

(54) INTELLIGENT INTERACTIVE-RHYTHMIC NEUROMUSCULAR REHABILITATION SYSTEM

(71) Applicant: HEALER TECH CO., LTD., Beijing (CN)

(72) Inventors: Shi CHENG, Beijing (CN); Li GE, Beijing (CN); Chen MIAO, Beijing (CN); Yifei REN, Beijing (CN)

(21) Appl. No.: 15/026,590

(22) PCT Filed: Aug. 18, 2015
(86) PCT No.: PCT/CN2015/087362

§ 371 Date: Mar. 31, 2016

(30) Foreign Application Priority Data
Sep. 16, 2014 (CN) 201410472127.3

(51) Int. Cl. A61B 5/00; A61B 5/04; A61B 5/0488

(52) U.S. Cl. CPC A61B 5/486; A61B 5/0488; A61B 5/0004; A61B 5/04012; A61B 5/7203; A61B 5/7264; A61B 5/7405; A61B 5/742; A61B 5/7455; A61B 2505/09; A61B 2560/0223

(57) ABSTRACT

The present invention provides an intelligent interactive-rhythmic neuromuscular rehabilitation system, comprising: a surface electromyography sensor configured to collect a user's electromyographic signal; a signal transmitter configured to receive and transmit the electromyographic signal; a rhythmic interaction module configured to generate and provide a specific rhythm to the user and receive the electromyographic signal, such that when the electromyographic signal matches the specific rhythm, a positive feedback is provided; otherwise, a negative feedback is provided. The intelligent interactive-rhythmic neuromuscular rehabilitation system enables a user to constantly strengthen a target muscle or muscle group by a rhythmic interaction module, and to be familiar with the voluntary control of target muscle by motor nerve control and suppressing improper muscle contraction, thereby achieving muscle balance; The system is characterized by its high interactivity, intelligence, and entertainment. It enhances the user's adherence to the long-term treatment, and makes user's self-treatment possible.

### FIELD OF THE INVENTION

The present invention relates to the field of medical device, and more specifically to an intelligent interactive-rhythmic neuromuscular rehabilitation system.

### BACKGROUND OF THE INVENTION

Biofeedback is a treatment concept of reflecting physiological activity of a human body using various kinds of medical devices such that a user obtains greater intuitive awareness of physiological function, thereby achieving free control of physiological activities that originally could not be voluntarily controlled by facilitating or inhibiting behaviors with positive or negative feedback. It has always been a leading-edge area of research how to apply biofeedback to enable a user to focus more on a treatment process and to adhere to a long-term treatment so as to achieve an optimal treatment outcome.

The existing neuromuscular rehabilitation system is generally used in a clinic or a hospital for rehabilitation, in which patients have to make an appointment and personally go to the facility to receive the treatment. Such a model wastes considerable time and manpower; besides, due to the tedium during the treatment process, the patient does not have a strong will to adhere to the long-term treatment. Consequently, it is less possible for patient's self-treatment. In addition, the existing neuromuscular rehabilitation system is not portable due to its bulkiness and heavy dependence on wired USB transmission among the parts. In an actual treatment process, due to the above deficiencies, most patients cannot adhere to and finally have to give up treatment, thereby greatly interfering treatment efficacy.

In addition, because a common practice of patient care is one clinician overseeing one patient, and its efficiency is also very low.

### SUMMARY OF THE INVENTION

To this end, the present invention provides a novel neuromuscular rehabilitation system that may solve at least part of the above problems.

The present invention provides an intelligent interactive-rhythmic neuromuscular rehabilitation system, comprising: a surface electromyography sensor configured to collect a user's electromyographic signal; a signal transmitter configured to receive and transmit the electromyographic signal; a rhythmic interaction module configured to generate and provide a specific rhythm to the user and receive the electromyographic signal, such that when the electromyographic signal matches the specific rhythm, a positive feedback is provided; otherwise, a negative feedback is provided.

According to one embodiment of the present invention, there further comprises: a signal processor configured to receive the electromyographic signal from the surface electromyography sensor and perform smooth processing to the electromyographic signal.

According to one embodiment of the present invention, the electromyographic signal is subjected to smooth processing in the following manner, deriving output y:

$$y = \frac{{\int }_{0}^{{t}_{n}} x d t}{ndt} ,$$

where x denotes a sample of the input signal, n denotes an amount of samples, dt denotes a sampling time interval, while tₙ denotes a total sampling time.

According to one embodiment of the present invention, the rhythmic interaction module is calibrated in the following manner calculating a range of steady-state value, wherein the range of steady-state value is between (1−b %) a to (1+b %) a, where b % is an environmental noise tolerance, while a is a mean value when muscle is relaxing; and setting an electromyographic signal with a value larger than (1+b %) a as 1, and setting an electromyographic signal with a value less than (1+b %) a as 0.

According to one embodiment of the present invention, the rhythmic interaction module is calibrated in the following manner calculating a range of steady-state value, wherein the range of steady-state value is between (1−b %) a to (1+b %) a, where b % is an environmental noise tolerance, while a is a mean value when muscle is relaxing; calculating an absolute value of the electromyographic signal; calculating a ratio of a difference between the absolute value of the electromyographic signal and an upper bound (1+b %) a of the steady-state value threshold to the upper bound (1+b %) a of the steady-state value threshold; and calculating a logarithm of the ratio.

According to one embodiment of the present invention, there further comprises a remote monitoring device configured to at least receive the positive feedback or negative feedback from the rhythmic interaction module so as to remotely monitor user's muscle activity.

According to one embodiment of the present invention, the rhythm comprises a plurality of interactive elements, a time interval between adjoining interactive elements being adjustable.

According to one embodiment of the present invention, the rhythm comprises at least one of a preliminary phase mode, an intermediary phase mode, and an advanced phase mode, wherein in the preliminary phase mode, the next interactive element is given only after the previous one is completed; in the intermediary phase mode, there is a relatively long interval between adjoining interactive elements, and each interactive element appears regularly; and in the advanced phase mode, there is a relatively short interval between adjoining interactive elements, and each interactive element appears irregularly.

According to one embodiment of the present invention, the rhythm has a form of at least one of audio, video, and tactile sense.

According to one embodiment of the present invention, the signal transmitter is a wireless transmitter.

The present invention further provides an intelligent-rhythm interactive-type electromyographic signal neuromuscular rehabilitation device, comprising: a module configured to generate and provide a specific rhythm to a user; a module configured to receive an electromyographic signal; and a module configured to provide a positive feedback when the electromyographic signal matches the specific rhythm and provide a negative feedback otherwise.

The intelligent interactive-rhythmic neuromuscular rehabilitation system in the present invention enables a user to constantly strengthen and build a target muscle or muscle group by a rhythmic interaction module, and to be familiar with the voluntary control of target muscle by motor nerve and suppress error contraction of the muscle, thereby achieving an effect of balanced countermeasuring the muscle; besides, it has an extremely high interactivity, intelligence, and fun. It raises the user's willingness in persistence in long-term treatment, and makes it possible for the user to perform self-treatment.

### BRIEF DESCRIPTION OF DRAWINGS

By reading the following detailed description of preferred embodiments, It will be much easier for general technician in this field to understand other advantages and benefits. The drawings are only for the purpose of illustrating preferred embodiments and should not be regarded as limitations to the present invention. Moreover, in the whole drawings, the same reference numbers are used for representing the same components. In the accompanying drawings, alphabetical labels after the reference numbers represent a plurality of same components; in general when they generally refer to these components, their last alphabetic labels will be omitted. In the drawings:

FIG. 1 shows a structural diagram of an intelligent interactive-rhythmic neuromuscular rehabilitation system according to the present invention;

FIG. 2 shows a structural diagram of a surface electromyography sensor;

FIG. 3-1 shows an unprocessed electromyographic signal waveform diagram;

FIG. 3-2 shows an electromyographic signal waveform diagram processed by smooth processing;

FIG. 4 shows a binary value waveform diagram converted from the processed electromyographic signal waveform diagram in FIG. 3-1;

FIG. 5 shows an electromyographic signal waveform diagram processed by taking logarithm; and

FIG. 6 shows a block diagram of an intelligent interactive-rhythmic neuromuscular rehabilitation system according to another embodiment of the present invention;

the meanings of each reference numerals in the drawings are specified as follows:

a surface electromyography sensor 10, an electrode sensor 11, a signal amplifying circuit 12, a signal full-wave rectifying circuit 13, a signal smoothing circuit 14, a signal processor 15, a signal transmitter 20, a rhythmic interaction module 30, a remote monitoring device 40, a module 100 configured to generate and provide a specific rhythm to a user, a module 200 configured to receive an electromyographic signal, and a module 300 configured to provide a positive feedback when the electromyographic signal matches the specific rhythm, otherwise provide a negative feedback.

### DETAILED DESCRIPTION OF THE PREFERRED EMBODIMENTS

Hereinafter, the present invention will be further described in conjunction with the accompanying drawings and specific embodiments.

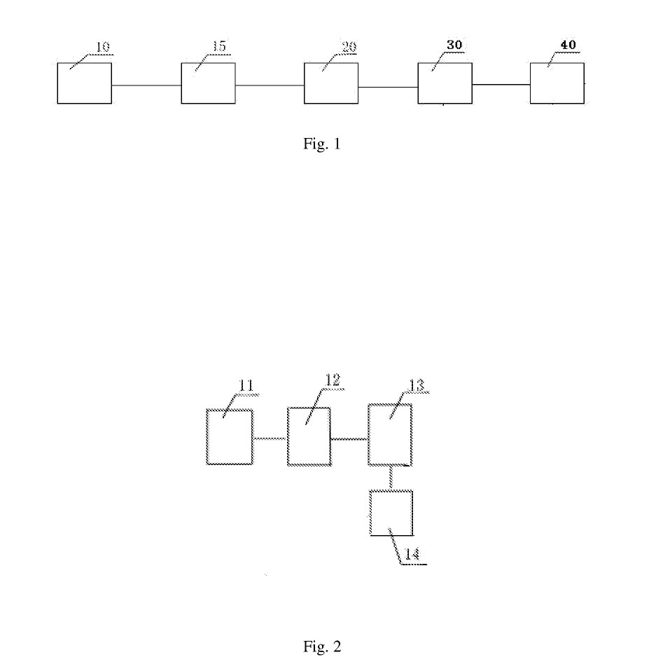

FIG. 1 shows a structural diagram of an intelligent interactive-rhythmic neuromuscular rehabilitation system according to the present invention. As shown in FIG. 1, the intelligent interactive-rhythmic neuromuscular rehabilitation system comprises a surface electromyography sensor 10, a signal processor 15, a signal transmitter 20, and a rhythmic interaction module 30, wherein the surface electromyography sensor 10 is configured to collect an electromyographic signal of a user, and the surface electromyography sensor signal collector 10 comprises an electrode sensor 11, a signal amplification circuit 12, a signal full-wave rectification circuit 13, and a signal smoothing circuit 14.

The signal transmitter 20 may be a wired transmitter or a wireless transmitter. The wireless transmitter, for example, may be via Bluetooth or WiFi, or be an infrared transmitter. The most important feature of Bluetooth is power saving. A button cell is able to support a Bluetooth device to work for several years thanks to the extremely low power consumption of Bluetooth. The main advantages of Bluetooth are: very low peak value, average and standby mode power consumption; low cost; wireless coverage enhancement; complete downward compatibility and low delay (APT-X). Therefore, the signal transmitter 20 is preferably via Bluetooth. In the intelligent interactive-rhythmic neuromuscular rehabilitation system, in the case that the electromyographic signal collected by the surface electromyography sensor 10 is acceptable, it may be directly transmitted to the signal transmitter 20; in the case that if eletromyographic signal collected by electromyography sensor 10 is not acceptable, the signal will be processed by the signal processor 15 and then transmitted to the signal transmitter 20, so that the intelligent interactive-rhythmic neuromuscular rehabilitation system preferably comprises the signal processor 15.

As shown in FIG. 2, the surface electromyography sensor signal collector 10 comprises an electrode sensor 11, a signal amplification circuit 12, a signal full-wave rectification circuit 13, and a signal smoothing circuit 14; the electrode sensor 11 being is connected to the signal amplification circuit 12; the electrode sensor 11 comprises a reference electrode, a muscle middle-end electrode, and a muscle terminal-end electrode; the electrode sensor 11 transmits a first electrical signal obtained from a body surface to the signal amplification circuit 12 that amplifies the first electrical signal and then transmits it to the signal full-wave rectification circuit 13; the signal full-wave rectification circuit 13 is connected to the signal smoothing circuit 14; the signal full-wave rectification circuit 13 converts alternative current to direct current, and transmits the direct current electrical signal to the signal smoothing circuit 14; and the signal smoothing circuit 14 smoothes the direct current electrical signal, and then convert the signal to square wave which becomes the second electrical signal, and is transmitted to signal processor 15.

The signal transmitter 20 is connected to the signal processor 15. The signal transmitter 20 is configured to receive and transmit the electromyographic signal; the rhythm rhythmic interaction module 30 is for generating a specific rhythm. The rhythm rhythmic interaction module 30 receives the electromyographic signal from the signal transmitter 20 and compares the electromyographic signal with the specific rhythm to determine whether they match each other or not; when the electromyographic signal matches the specific rhythm, a positive feedback is provided; and when the electromyographic signal does not match the specific rhythm, a negative feedback is provided.

The indication for the intelligent interactive-rhythmic neuromuscular rehabilitation system according to the present invention include: muscle imbalance, e.g., upper or lower crossed syndrome, and cervical instability (muscle group disorder surrounding the cervical spine); muscle weakness, e.g., patellofemoral pain syndrome, lumbar instability and cervical instability; and motor nerve control disorder, e.g., pelvic floor dysfunction, postpartum pelvic pain, incontinence and foot drop gait. The inventive intelligent interactive-rhythmic neuromuscular rehabilitation system enables a user to constantly strengthen a target muscle or a muscle group by a rhythmic interaction module, and to be familiar with the voluntary control of target muscle by facilitating motor nerve control and suppressing improper contraction of the muscle, thereby achieving muscle balance; besides, it is characterized by incomparable interactivity, intelligence, and entertainment. The system enhances patient's will to adhere to the long term treatment, and makes user's self-treatment possible.

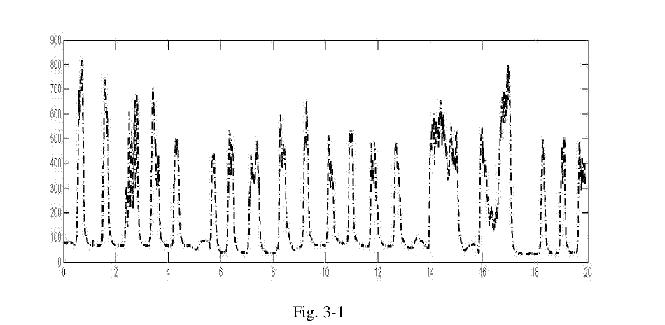

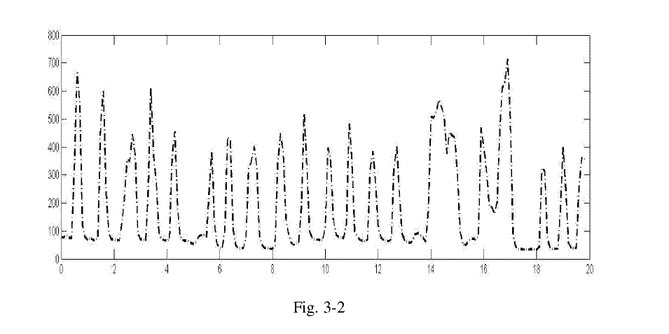

In the intelligent interactive-rhythmic neuromuscular rehabilitation system according to the present invention, the signal processor 15 is disposed between the surface electromyography sensor 10 and the signal transmitter 20; the signal processor 15 comprises an A/D converter and a digital signal processor. The signal processor 15 is configured to receive the electromyographic signal from the surface electromyography sensor 10. A second electrical signal is derived by amplifying, converting and smoothing the electromyographic signal and square wave conversion. Then, the second electrical signal is transmitted to the signal processor 15. The signal processor 15 is connected to the signal transmitter 20. The signal processor 15 converts the second electrical signal into a first digital signal. A second digital signal is derived after the first digital signal is derived by taking the algorithm of numerical integration and averaging. The specific processing method is provided below:

$$y = \frac{{\int }_{0}^{{t}_{n}} x d t}{ndt}$$

wherein x denotes a sample of the input signal, n denotes the number of samples, dt denotes a sampling time interval, and tₙ denotes a total sampling time. The electromyographic signal is smoothed based on the above algorithm, and then the output y is derived. FIG. 3-1 shows an unprocessed electromyographic signal waveform graph, and FIG. 3-2 shows a smooth processed electromyographic signal waveform graph, the smooth process effectively avoids a burst noise generated instantaneously and avoids incapability of accurately detecting the user's muscle control behavior accurately.

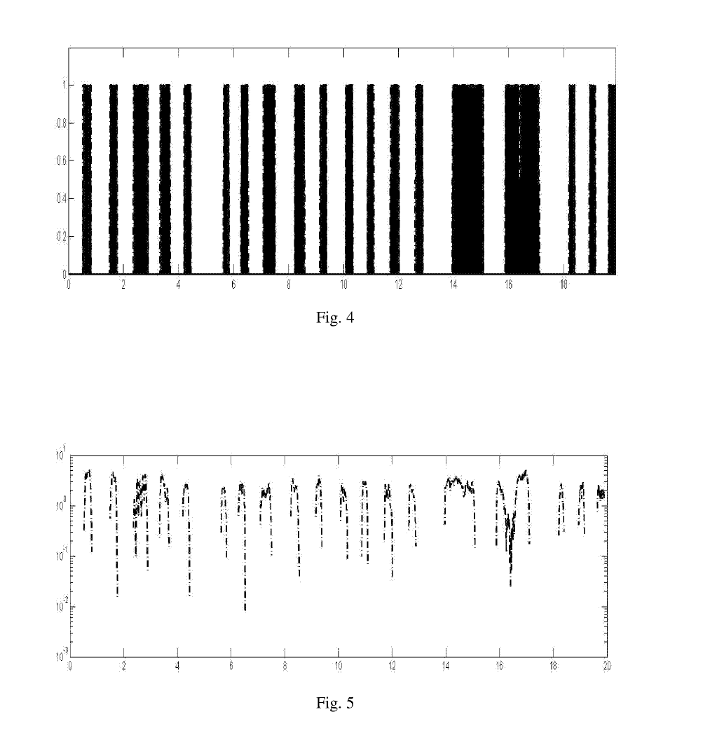

In the intelligent interactive-rhythmic neuromuscular rehabilitation system according to the present invention, the rhythmic interaction module 30 is calibrated in the following manner calculating a range of steady-state value, wherein the range of steady-state value is between (1−b %) a to (1+b %) a, where b % is an environmental noise tolerance, while a is a mean value when muscle is relaxing; setting an electromyographic signal with a value larger than (1+b %) a as 1, and setting an electromyographic signal with a value less than (1+b %) a as 0. The range of steady-state value refers to a numerical range when the user's muscle is relaxing. Without the range of steady-state value, the system cannot identify a muscle contraction. Before the beginning of the treatment, the muscle is in a relaxing state, and rhythmic interaction module 30 instantaneously starts collecting data and analyzing, finally obtaining a mean value a and an environmental noise tolerance b % when the muscle is relaxing; Consequently when the muscle is relaxing, the received data will vibrate in the range between (1−b %) and (1+b %) a, i.e., vibrating in the steady-state value range. During the muscle contraction and relaxation process of the user, an electromyographic signal will be generated. Consecutive numerical values of the received electromyographic signal are converted into a binary value. The binary value 1 denotes correct, while 0 denotes incorrect. For an electromyographic signal with a value greater than (1+b %) a, it is set to 1; for an electromyographic signal with a valueless than (1+b %) a, it is set to 0. FIG. 4 shows a binary value waveform diagraph converted from the unprocessed electromyographic signal waveform in FIG. 3-1.

In the intelligent interactive-rhythmic neuromuscular rehabilitation system according to the present invention, the rhythmic interaction module 30 is calibrated in the following manner calculating a range of steady state value, wherein the range of steady state value is between (1−b %) a and (1+b %) a, where b % is an environmental noise tolerance, while a is a mean value when muscle is relaxing; calculating the absolute value of the electromyographic signal; calculating a ratio of a difference between the absolute value of the electromyographic signal and an upper bound (1+b %) a of the steady-state value threshold to divided by the upper bound (1+b %) a of the steady-state value threshold; and calculating the logarithm of the ratio. FIG. 5 shows an electromyographic signal waveform diagram resulting from taking the logarithm on the unprocessed electromyographic signal waveform diagram in FIG. 3-1.

In the intelligent interactive-rhythmic neuromuscular rehabilitation system according to the present invention, there further comprises a remote monitoring device 40. As shown in FIG. 1, the remote monitoring device 40 is connected to the rhythmic interaction module 30. The remote monitoring device 40 is configured to at least receive the positive feedback or negative feedback from the rhythmic interaction module 30 so as to remotely monitor the user's muscle activity. A physician or a therapist may know the user's muscle recovery process through a feedback result on the remote monitoring device 40, and thereby formulating different treatment plan.

In the intelligent interactive-rhythmic neuromuscular rehabilitation system according to the present invention, the rhythm comprises a plurality of interactive elements, and a time interval between adjoining interactive elements is adjustable.

A plurality of embodiments may be designed based on the rhythms and rhythm patterns provided in the present invention. Therefore, the preferred embodiments are only exemplary illustrations of the specific implementation manners of the present invention, not constituting a limitation to the scope of the present invention. In order to specifically describe the present invention, the following embodiments are selected for exemplary illustration.

### Embodiment 1

FIG. 1 shows a structural diagram of an intelligent interactive-rhythmic neuromuscular rehabilitation system according to the present invention. As shown in FIG. 1, the intelligent interactive-rhythmic neuromuscular rehabilitation system according to the present invention comprises a surface electromyography sensor 10, a signal processor 15, a signal transmitter 20, and a rhythmic interaction module 30, wherein the surface electromyography sensor 10 is configured to collect an electromyographic signal of a user; the signal processor 15 is configured to receive the electromyographic signal from the surface electromyography sensor 10, processes the electromyographic signal to obtain a smoothed electromyographic signal, and then transmits the smoothed electromyographic signal to the signal transmitter 20; and the rhythmic interaction module 30 receives the electromyographic signal from the signal transmitter 20. The rhythmic interaction module 30 is for generating a specific rhythm, and compares the electromyographic signal with the specific rhythm to determine whether they match, such that when the electromyographic signal matches the specific rhythm, a positive feedback is provided; and when the electromyographic signal does not match the specific rhythm, a negative feedback is provided.

The rhythm comprises multiple interactive elements, and the time interval between adjoining interactive elements is adjustable, such that a reasonable interval may be set based on different recovery phases of a user's muscle. The rhythm is in a preliminary phase mode, the rhythm is in the form of an audio or a video or a tactile form. The audio is any audible audio, the video is any viewable video, and the tactile form is any sensible tactile stimuli, e.g., vibration. In the present embodiment, the rhythm is in the form of an audio. The preliminary phase mode is a phase where the user cannot correctly control muscle behaviors, the rhythm is in the form of a musical audio, a specific beat of the music is referred to as an interactive element, e.g., a “tick tock” beat. Upon hearing the “tick tock” beat, the user controls the muscle to react. The user's muscle reaction to the rhythm is non-prompt. The user is allowed to interact with the following interactive element only if he/she completes the current interactive element. The electromyographic signal is compared with the specific rhythm to determine whether the two match each other or not. When the electromyographic signal matches the specific rhythm, a positive feedback is provided; otherwise, a negative feedback is provided. There are multiple forms of providing the positive feedback or negative feedback. For example, in the case of providing a positive feedback, the rhythmic interaction module 30 will add a score, while in the case of providing a negative feedback, the rhythmic interaction module 30 will deduct a score, which enhances the entertainment and may encourage the user to constantly pursue a higher score. The positive feedback or negative feedback is transmitted to the remote monitoring device 40 so as to remotely monitor the user's muscle activity. In other cases, the audio may be a buzz sound. There are many forms of audio presentations, not limited to the music or buzz sound mentioned in the present embodiment. Besides, the rhythm may also be in a tactile form, such that the user can react when sensing a tactile change.

### Embodiment 2

FIG. 1 shows a structural diagram of an intelligent interactive-rhythmic neuromuscular rehabilitation system according to the present invention. As shown in FIG. 1, the intelligent interactive-rhythmic neuromuscular rehabilitation system according to the present invention comprises a surface electromyography sensor 10, a signal processor 15, a signal transmitter 20, and a rhythmic interaction module 30, wherein the surface electromyography sensor 10 is configured to collect an electromyographic signal of a user, the signal processor 15 is configured to receive the electromyographic signal from the surface electromyography sensor 10, processes the electromyographic signal to obtain a smoothed electromyographic signal, and then transmits the smoothed electromyographic signal to the signal transmitter 20; and the rhythmic interaction module 30 receives the electromyographic signal from the signal transmitter 20. The rhythmic interaction module 30 is for generating a specific rhythm, and compares the electromyographic signal with the specific rhythm to determine whether they match, such that when the electromyographic signal matches the specific rhythm, a positive feedback is provided, and when the electromyographic signal does not match the specific rhythm, a negative feedback is provided.

The rhythm comprises a plurality of interactive elements, and the time interval between adjoining interactive elements is adjustable, such that a reasonable interval may be set based on different recovery phases of a user's muscle. The rhythm is in an intermediate phase mode, and it is in the form of an audio or a video, or in a tactile form. The audio is any audible audio, and the video is any viewable video. In the present embodiment, the rhythm is in the form of an audio. The intermediate phase mode is a phase where the user cannot skillfully control his/her muscle behaviors yet, and the rhythm is in the form of a musical audio. A specific beat of the music is referred to as an interactive element, e.g., a “tick tock” beat. The rhythm is a relatively slow regular rhythm, e.g., there is a “tick tock” rhythm every 5 seconds; and upon hearing the “tick tock” beat, the user needs to react promptly, i.e., controlling the muscle to contract or relax, thereby requiring the user to interact once every 5 seconds. The electromyographic signal is compared with the specific rhythm to determine whether the two match each other or not. When the electromyographic signal matches the specific rhythm, a positive feedback is provided; otherwise, a negative feedback is provided. There are multiple forms of providing the positive feedback or negative feedback. For example, in the case of providing positive feedback, the rhythmic interaction module 30 will automatically add a score, while in the case of providing a negative feedback, the rhythmic interaction module 30 will automatically deduct the score, which enhances the entertainment. The positive feedback or negative feedback is transmitted to the remote monitoring device 40 so as to remotely monitor the user's muscle activity. The audio may also be a buzz sound. There are many forms of audio representation, not limited to the music or buzz sound mentioned in the present embodiment. Besides, the rhythm may also be a tactile form, such that the user can react when sensing a tactile change.

### Embodiment 3

FIG. 1 shows a structural diagram of an intelligent interactive-rhythmic neuromuscular rehabilitation system according to the present invention. As shown in FIG. 1, the intelligent interactive-rhythmic neuromuscular rehabilitation system according to the present invention comprises a surface electromyography sensor 10, a signal processor 15, a signal transmitter 20, and a rhythmic interaction module 30, wherein the surface electromyography sensor 10 is configured to collect an electromyographic signal of a user; the signal processor 15 is configured to receive the electromyographic signal from the surface electromyography sensor 10, and processes the electromyographic signal to obtain a smoothed electromyographic signal, and then transmits the smoothed electromyographic signal to the signal transmitter 20. The rhythmic interaction module 30 receives the electromyographic signal from the signal transmitter 20; and the rhythmic interaction module 30 is for generating a specific rhythm, and compares the electromyographic signal with the specific rhythm to determine whether they match, such that when the electromyographic signal matches the specific rhythm, a positive feedback is provided, and when the electromyographic signal does not match the specific rhythm, a negative feedback is provided.

The rhythm comprises a plurality of interactive elements, and the time interval between adjoining interactive elements is adjustable, such that a reasonable interval may be set based on different recovery phases of a patient's muscle. The rhythm is in an advanced phase mode, and it is in the form of an audio or a video, or in a tactile form. The audio is any audible audio, and the video is any viewable video. In the present embodiment, the rhythm is in the form of an audio. The advanced phase mode is a phase where the user can skillfully control his/her muscle behaviors, and the form of the rhythm is a musical audio. A specific beat of the music is referred to as an interactive element, e.g., a “tick tock” beat. The rhythm is a very fast regular rhythm, e.g., the “tick tock” beat appears randomly; and upon hearing the “tick tock” beat, the user needs to react immediately, i.e., controlling the muscle to make an action. The electromyographic signal is compared with the specific rhythm to determine whether the two match each other or not. When the electromyographic signal matches the specific rhythm, a positive feedback is provided; otherwise, a negative feedback is provided. There are multiple forms of providing the positive feedback or negative feedback. For example, in the case of providing a positive feedback, the rhythmic interaction module 30 will automatically add a score, while in the case of providing a negative feedback, the rhythmic interaction module 30 will automatically deduct a score, which enhances entertainment. The positive feedback or negative feedback is transmitted to the remote monitoring device 40 so as to remotely monitor the user's muscle activity. The audio may also be a buzz sound. There are many forms of audio representation, not limited to the music or buzz sound mentioned in the present embodiment. Besides, the rhythm may also be in a tactile form, such that the user can react when sensing a tactile change.

### Embodiment 4

FIG. 1 shows a structural diagram of an intelligent interactive-rhythmic neuromuscular rehabilitation system according to the present invention. As shown in FIG. 1, the intelligent interactive-rhythmic neuromuscular rehabilitation system according to the present invention comprises a surface electromyography sensor 10, a signal processor 15, a signal transmitter 20, and a rhythmic interaction module 30, wherein the surface electromyography sensor 10 is configured to collect an electromyographic signal of a user; the signal processor 15 is configured to receive the electromyographic signal from the surface electromyography sensor 10, processes the electromyographic signal to obtain a smoothed electromyographic signal, and then transmits the smoothed electromyographic signal to the signal transmitter 20; and the rhythmic interaction module 30 receives the electromyographic signal from the signal transmitter 20. The rhythmic interaction module 30 is for generating a specific rhythm, and compares the electromyographic signal with the specific rhythm to determine whether they match, such that when the electromyographic signal matches the specific rhythm, a positive feedback is provided; and when the electromyographic signal does not match the specific rhythm, a negative feedback is provided.

The rhythm comprises multiple interactive elements, and the time interval between adjoining interactive elements is adjustable, such that a reasonable interval may be set based on different recovery phases of a patient's muscle. The rhythm is a preliminary phase mode, and it is in the form of an audio or a video or in a tactile form. The audio is any audible audio, and the video is any viewable video. In the present embodiment, the rhythm is presented in a video form. The preliminary phase mode is a phase where the user cannot correctly control muscle behaviors yet, the form of the rhythm is a video of jumping over obstacles in the Parkour game application, and each obstacle is referred to as an interactive element. Upon viewing an obstacle displayed by the rhythmic interaction module 30, the user needs to react by controlling the muscle to contract or relax, and after this action is completed, the next obstacle is displayed. The electromyographic signal of the user's muscle activity is collected by the surface electromyography sensor 10. The electromyographic signal is compared with the specific rhythm to determine whether the two match each other or not. When the electromyographic signal matches the specific rhythm, a positive feedback is provided; otherwise, a negative feedback is provided. There are multiple forms of providing the positive feedback or negative feedback. For example, in the case of providing a positive feedback, the rhythmic interaction module 30 will automatically add a score, while in the case of providing a negative feedback, the rhythmic interaction module 30 will automatically deduct a score, which enhances entertainment. The positive feedback or negative feedback is transmitted to the remote monitoring device 40 so as to remotely monitor the user's muscle activity. There are many forms of video representation, not limited to jumping over the obstacles in the Parkour game application mentioned in the present embodiment. Besides, the rhythm may also be in a tactile form, such that the user can react when sensing a tactile change.

### Embodiment 5

FIG. 1 shows a structural diagram of an intelligent interactive-rhythmic neuromuscular rehabilitation system according to the present invention. As shown in FIG. 1, the intelligent interactive-rhythmic neuromuscular rehabilitation system according to the present invention comprises a surface electromyography sensor 10, a signal processor 15, a signal transmitter 20, and a rhythmic interaction module 30, wherein the surface electromyography sensor 10 is configured to collect an electromyographic signal of a user; the signal processor 15 is configured to receive the electromyographic signal from the surface electromyography sensor 10, processes the electromyographic signal to obtain a smoothed electromyographic signal, and then transmits the smoothed electromyographic signal to the signal transmitter 20; and the rhythmic interaction module 30 receives the electromyographic signal from the signal transmitter 20. The rhythmic interaction module 30 is for generating a specific rhythm, and compares the electromyographic signal with the specific rhythm to determine whether they match, such that when the electromyographic signal matches the specific rhythm, a positive feedback is provided; and when the electromyographic signal does not match the specific rhythm, a negative feedback is provided.

The rhythm comprises multiple interactive elements, and the time interval between adjoining interactive elements is adjustable, such that a reasonable interval may be set based on different recovery phases of a patient's muscle. The rhythm is an intermediary phase mode, and it is in the form of an audio or video or in a tactile form. The audio is any audible audio, and the video is any viewable video. In the present embodiment, the rhythm is in the form of a video. The intermediary phase mode is a phase where the user cannot skillfully control muscle behaviors yet. The rhythm is a slow regular rhythm, and the form of the rhythm is selected as a video of jumping over obstacles in the Parkour game application, where each obstacle is referred to as an interactive element. Each obstacle appears regularly in the video, and there is a relatively long interval time; upon viewing an obstacle displayed by the rhythmic interaction module 30, the user needs to react i.e., controlling the muscle to contract or relax; and when the following obstacles are shown regularly, the user reacts promptly. The electromyographic signals of the user's muscle control are collected by the surface electromyography sensor 10. The electromyographic signal is compared with the specific rhythm to determine whether the two match each other or not. When the electromyographic signal matches the specific rhythm, a positive feedback is provided; otherwise, a negative feedback is provided. There are multiple forms of providing the positive feedback or negative feedback. For example, in the case of providing a positive feedback, the rhythmic interaction module 30 will automatically add a score, while in the case of providing a negative feedback, the rhythmic interaction module 30 will automatically deduct a score, which enhances entertainment. The positive feedback or negative feedback is transmitted to the remote monitoring device 40 so as to remotely monitor the user's muscle activity. There are many forms of audio representation, not limited to jumping over the obstacles in the Parkour game application mentioned in the present embodiment. Besides, the rhythm may also be in a tactile form, such that the user can react when tactilely sensing a change.

### Embodiment 6

FIG. 1 shows a structural diagram of an intelligent interactive-rhythmic neuromuscular rehabilitation system according to the present invention. As shown in FIG. 1, the intelligent interactive-rhythmic neuromuscular rehabilitation system according to the present invention comprises a surface electromyography sensor 10, a signal processor 15, a signal transmitter 20, and a rhythmic interaction module 30, wherein the surface electromyography sensor 10 is configured to collect an electromyographic signal of a user; the signal processor 15 is configured to receive the electromyographic signal from the surface electromyography sensor 10, processes the electromyographic signal to obtain a smoothed electromyographic signal, and then transmits the smoothed electromyographic signal to the signal transmitter 20; and the rhythmic interaction module 30 receives the electromyographic signal from the signal transmitter 20. The rhythmic interaction module 30 is for generating a specific rhythm, and compares the electromyographic signal with the specific rhythm to determine whether they match, such that when the electromyographic signal matches the specific rhythm, a positive feedback is provided; and when the electromyographic signal does not match the specific rhythm, a negative feedback is provided.

The rhythm comprises multiple interactive elements, and the time interval between adjoining interactive elements is adjustable, such that a reasonable interval may be set based on different recovery phases of a patient's muscle. The rhythm is in an advanced phase mode, and it is in the form of an audio or a video or in a tactile form. The audio is any audible audio, and the video is any viewable video. In the present embodiment, the rhythm is in the form of a video. The advanced phase mode is a phase where the user can skillfully control muscle behaviors. The rhythm is a fast irregular rhythm, and the form of the rhythm is a video of jumping over obstaclesobstacle in the Parkour game application, where each obstacle is referred to as an interactive element. Each obstacle appears irregularly in the video, and there is a short interval time. Upon viewing an obstacle displayed by the rhythmic interaction module 30, the user needs to react i.e., controlling the muscle to contract or relax, and when the following obstacles are shown irregularly, the user reacts immediately. The electromyographic signal of the user's the muscle control is collected by the surface electromyography sensor 10. The electromyographic signal is compared with the specific rhythm to determine whether the two match each other or not. When the electromyographic signal matches the specific rhythm, a positive feedback is provided; otherwise, a negative feedback is provided. There are multiple forms of providing the positive feedback or negative feedback. For example, in the case of providing a positive feedback, the rhythmic interaction module 30 will automatically add a score, while in the case of providing a negative feedback, the rhythmic interaction module 30 will automatically deduct a score, which enhances entertainment. The positive feedback or negative feedback is transmitted to the remote monitoring device 40 so as to remotely monitor the user's muscle activity. Besides, the rhythm may also be in a tactile form, such that the user reacts when sensing a tactile change.

There are many forms of audio representation, not limited to jumping over the obstacles in the Parkour game application mentioned in the present embodiment. The intelligent interactive-rhythmic neuromuscular rehabilitation system enables a user to constantly strengthen a target muscle or muscle group by the rhythmic interaction module 30, and to be familiar with the voluntary control of target muscle by facilitating motor nerve control and suppressing improper muscle contraction, thereby achieving muscle balance; besides, it is characterized by interactivity, intelligence, and entertainment. The system enhances user's adherence to long-term treatment, and makes user's self-treatment possible.

The inventive intelligent interactive-rhythmic neuromuscular rehabilitation system enables a user to constantly strengthen a target muscle or muscle group by a rhythmic interaction module, and to be familiar with the voluntary control of target muscle by facilitating motor nerve control and suppressing improper muscle contraction, thereby achieving muscle balance; besides, it is characterized by interactivity, intelligence, and entertainment. The system enhances user's adherence to long-term treatment. Further, through detecting the user's treatment process by a remote monitoring device, the user does not have to go to a clinic or a hospital for receiving treatment, which saves much time and labor of the user.

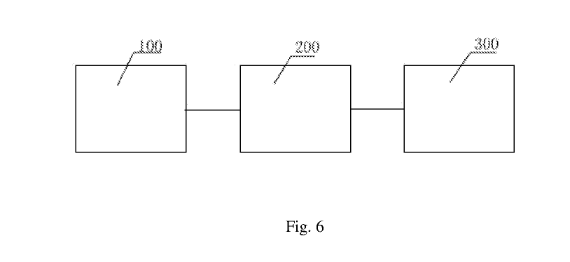

The present invention further provides an intelligent-rhythm interactive-type electromyographic signal neuromuscular rehabilitation device, comprising: a module 100 configured to generate and provide a specific rhythm to a user; a module 200 configured to receive an electromyographic signal; and a module 300 configured to give a positive feedback when the electromyographic signal matches the specific rhythm, and otherwise give a negative feedback. FIG. 6 shows a block diagram of an intelligent-rhythm interactive-type electromyographic signal neuromuscular rehabilitation device according to another embodiment of the present invention.

It should be understood that each block in the block diagrams and flow diagrams, as well as a combination of the blocks in the block diagrams and flow diagrams, may be implemented by a circuit, processor, software, or various combinations of them, and may also be implemented by various modules including computer program instructions. These computer program instructions may be loaded on a general-purpose computer, a dedicated computer, or other programmable data processing device so as to produce a machine, such that an instruction performed on the computer or other programmable data processing device creates a module for performing a specified function in one or more flow diagram blocks.

These computer program instructions may also be stored in a computer-readable memory that may boot a computer or other programmable data processing device so as to function in a specific manner; the instructions stored in the computer-readable memory manufacture a product of a computer-readable instruction comprising functions specified in one or more flow diagram blocks. The computer program instruction may also be loaded onto the computer or other programmable data processing device such that a series of operation steps are executed on the computer or other programmable data processing device, thereby generating a computer-implemented process, and further the instruction executed on the computer or other programmable data processing device provides steps for implementing the specified functions in one or more flow diagram blocks.

Therefore, the blocks in the block diagrams and flow diagrams support a combination of modules for executing specified functions, a combination of steps for executing specified functions, and a combination of program instruction modules for executing specified functions. It should also be understood that each block in the block diagrams and flow diagrams, as well as a combination of blocks in the block diagrams and flow diagrams may be implemented by a hardware-based dedicated computer system for executing specified functions or steps, or implemented by a combination of dedicated hardware and computer instructions.

Those skilled in the art involved in these embodiments and benefited by studying the above described and associated figures will be aware of many modifications and other embodiments of the present invention disclosed here. Therefore, it should be understood that the present invention is not limited to the preferred embodiments disclosed here, but intended to include the modifications and other embodiments within the scope of the appended claims. Although specific terms are adopted here, they are only used in a general sense and description sense, not used for limitative purposes.

It should be noted that the above embodiments intend to illustrate, rather than limit the present invention. Moreover, without departing from the scope of the appended claims, those skilled in the art may design alternative embodiments. In the claims, no reference numerals included in the parentheses should constitute limitations to the claims. The word “comprise” should not exclude the elements or steps not listed in the claims. The word “a” or “one” before an element does not exclude a plurality of such elements. The present invention may be implemented by hardware including several different elements as well as an appropriately programmed computer. In the claim with several modules, some of the modules may be specifically embodied by one hardware device. Using of words such as first, second, and third does not represent any sequence. These words may be interpreted as names.

1. An intelligent interactive-rhythmic neuromuscular rehabilitation system, comprising:
a surface electromyography sensor configured to collect a user's electromyographic signal;
a signal transmitter configured to receive and transmit the electromyographic signal;
a rhythmic interaction module configured to generate and provide a specific rhythm to the user and receive the electromyographic signal, such that when the electromyographic signal matches the specific rhythm, a positive feedback is provided, otherwise, a negative feedback is provided.

2. The intelligent interactive-rhythmic neuromuscular rehabilitation system according to claim 1, further comprising:
a signal processor configured to receive the electromyographic signal from the surface electromyography sensor and smooth the signal.

3. The intelligent interactive-rhythmic neuromuscular rehabilitation system according to claim 2, wherein the smooth processing of the eletromyographic signal subjects to the following manner, deriving output y:
$$y = \frac{{\int }_{0}^{{t}_{n}} x d t}{ndt} ,$$
where x denotes a sample of the input signal, n denotes an amount of samples, dt denotes a sampling time interval, and to denotes a total sampling time.

4. The intelligent interactive-rhythmic neuromuscular rehabilitation system according to claim 1, wherein the rhythmic interaction module is calibrated in the following manner:
calculating a range of steady-state value, wherein the range of steady-state value is between (1−b %) a and (1+b %) a, where b % is an environmental noise tolerance, and a is an average when muscle is relaxing;
setting an electromyographic signal with a value greater than (1+b %) a as 1, and setting an electromyographic signal with a value less than (1+b %) a as 0.

5. The intelligent interactive-rhythmic neuromuscular rehabilitation system according to claim 1, wherein the rhythmic interaction module is calibrated in the following manner:
calculating a steady-state value range, wherein the steady-state value range is between (1−b %) a to (1+b %) a, where b % is an environmental noise tolerance, while a is a mean value when muscle is relaxing;
calculating the absolute value of the electromyographic signal;
calculating a ratio of a difference between the absolute value of the electromyographic signal and an upper bound (1+b %) a of the steady-state value threshold divided by the upper bound (1+b %) a of the tolerance of steady-state value; and
calculating a logarithm of the ratio.

6. The intelligent interactive-rhythmic neuromuscular rehabilitation system according to claim 1, wherein there further comprises a remote monitoring device configured to at least receive the positive feedback or negative feedback from the rhythmic interaction module so as to remotely monitor user's muscle activity.

7. The intelligent interactive-rhythmic neuromuscular rehabilitation system according to claim 1, wherein the rhythm comprises a plurality of interactive elements, and a time interval between adjoining interactive elements is adjustable.

8. The intelligent interactive-rhythmic neuromuscular rehabilitation system according to claim 7, wherein the rhythm comprises at least one of a preliminary phase mode, an intermediary phase mode, and an advanced phase mode, wherein,
in the preliminary phase mode, the next interactive element is given only after the previous one is completed;
in the intermediary phase mode, there is a relatively long interval between adjoining elements, and each interactive element appears regularly;
in the advanced phase mode, there is a relatively short interval between adjoining interactive elements, and each interactive element appears irregularly.

9. The intelligent interactive-rhythmic neuromuscular rehabilitation system according to claim 1, wherein the rhythm has a form of at least one of audio, video, and tactile sense.

10. The intelligent interactive-rhythmic neuromuscular rehabilitation system according to claim 1, wherein the signal transmitter is a wireless transmitter.

11. An intelligent-rhythm interactive-type electromyographic signal neuromuscular rehabilitation device, comprising:
a module configured to generate and provide a specific rhythm to a user;
a module configured to receive an electromyographic signal; and
a module configured to give a positive feedback when the electromyographic signal matches the specific rhythm, and otherwise give a negative feedback.
